(19) United States
(12) Patent Application Publication
(10) Pub. No.: US 2019/0142523 A1
(43) Pub. Date: May 16, 2019

(54) ENDOSCOPE-LIKE DEVICES COMPRISING SENSORS THAT PROVIDE POSITIONAL INFORMATION

(71) Applicant: MEDIGUS Ltd., Omer (IL)

(72) Inventor: Amir GOVRIN, Ramat Gan (IL)

(21) Appl. No.: 16/092,262

(22) PCT Filed: Jun. 1, 2017
(86) PCT No.: PCT/IL2017/050610

§ 371 Date: Oct. 9, 2018

(30) Foreign Application Priority Data
Jun. 6, 2016 (IL) 246068

(51) Int. Cl. A61B 34/20; A61B 1/04; A61B 1/00; A61B 1/005

(52) U.S. Cl. CPC A61B 34/20; A61B 1/04; A61B 1/00009; A61B 1/0051; A61B 2034/2048; A61B 1/00006

(57) ABSTRACT

Disclosed are a system and method to accurately determine the location and orientation of a part of an elongated insertion shaft of an endoscope-like device or of an object located on or in the elongated insertion shaft when the elongated insertion shaft is inserted into a lumen or cavity of a human or animal body or into the interior of a manufactured object such that direct visual observation of the part of the elongated insertion shaft or of the object located on or in the elongated insertion shaft is blocked.

### FIELD OF THE INVENTION

The invention is from the field of devices having elongated insertion shafts that are inserted into cavities for inspection, diagnosis, and treatment. Specifically the invention is related to methods of accurately locating within the cavity the position and orientation of a part of the elongated shaft or a component on the elongated shaft.

### BACKGROUND OF THE INVENTION

A conventional endoscope is illustrated in FIG. 1. The endoscope, generally indicated at 40, is provided with a handle or control section 41 provided with suction valves, locks, switches, etc., switches 42-45 being marked for illustration purposes. It also comprises a connector section 46 used to connect air and water inlets, light guides, etc., the light guide being indicated at 47, for illustration purposes. The insertion tube 48 consists of three separate sections: a flexible portion 49, an articulation section 50 and a distal tip 51. Some endoscopes do not comprise an articulation section and the entire insertion section is comprised of a flexible tube ending with a distal tip. In modern endoscopes the front face of the distal tip 51 typically comprises an objective lens that focuses light on a solid state imaging sensor, illumination sources, and the outlets of tubes for air and/or water used for insufflation and/or to clean the objective lens, and the outlets of one or more working channels that are used to deliver diagnostic or surgical tools through the insertion tube.

One of the difficulties encountered by surgeons during performance of medical procedures using endoscopes, laparoscopes, catheters, and delivery systems that have flexible insertion tubes is to know the exact location of the various parts of the device within the patient's body. This is a lesser problem, but it still is present in many situations when the insertion tube is rigid of a flexible insertion tube in a straight configuration since rotation of the handle section about the longitudinal axis of the endoscope by X degrees is accompanied by a corresponding rotation of the distal tip by X degrees, so that the surgeon knows the direction the visualization means is pointing and that the orientation of his instruments has to be the same as the orientation of the handle section. However, if the flexible part of the insertion section, either outside of the body of the patient or within the body cavity is curved, or the articulation section of either a flexible or rigid insertion shaft is activated to bend the distal part of the insertion section, then there is no longer an accurate correlation between the angle at which the handle rotates and the angle at which the distal tip rotates.

The most common method used today to overcome the problem is to use the visualization means of the device to locate anatomical structures to give an indication of where the distal tip is within the body cavity. The problem with this solution is that frequently part or all of the field of view is blocked either by nearby tissue or the lens becomes covered with fluids or debris that partially or totally renders the visualization means ineffective. Another method used to overcome the problem is fluoroscopy. This method has the disadvantage that the patient is subjected to the potential health risks that accompany exposure to radiation.

Another difficulty encountered by users whether using an endoscope or laparoscope with a rigid or flexible insertion section is that in the process of manipulating all of the levers and other controls on the handle while observing the images from the visualization means on a nearby display screen he/she can inadvertently move or rotate the handle of the device, which results in moving the distal tip from its intended location or orientation. The surgeon may be completely unaware of this movement which in some cases may have serious consequences.

It is therefore a purpose of the present invention to provide accurate positional information regarding the orientation of different parts of endoscopic and laparoscopic medical devices having flexible insertion shafts or rigid insertion shafts having moveable parts relative to other parts of the device and to the outside world.

It is another purpose of the present invention to provide a means for informing a surgeon if he moves his/her hand that holds the handle section or if the patient moves while performing a procedure with an endoscope or laparoscope having either a rigid or flexible insertion section.

Further purposes and advantages of this invention will appear as the description proceeds.

### SUMMARY OF THE INVENTION

In a first aspect the invention is a method to accurately determine the location and orientation of a part of an elongated insertion shaft of an endoscope-like device or of an object located on or in the elongated insertion shaft when the elongated insertion shaft is inserted into a lumen or cavity of a human or animal body or into the interior of a manufactured object such that direct visual observation of the part of the elongated insertion shaft or of the object located on or in the elongated insertion shaft is blocked. The method comprises:
a) attaching one or more rotation, position, motion, or angle sensors at one or more locations on the device;
b) sending via a wired or wireless communication network digital or analog signals produced by the sensors to a controller or microprocessor;
c) employing software and/or circuitry in the controller or microprocessor to determine information relating to motion, orientation, or rotation from the digital or analog signals produced by the sensors;
d) sending the information relating to motion, orientation, or rotation from the controller or microprocessor via a wired or wireless communication network to a display unit configured to generate audio and tactile signals and visual images that provide the operator with accurate positional information regarding different parts of the device relative to other parts of the device and to the outside world.

In embodiments of the method the sensors are selected from the group comprising: accelerometers, digital gyroscopes, solid state magnetic compasses, angle sensors and RFID tags.

In embodiments of the method the device is one of an endoscope, a laparoscope, a catheter, and a delivery system, the device configured for use in a medical application.

In embodiments of the method the device is a borescope or an endoscope, the device configured for use in an industrial application.

In embodiments of the method output signals from the sensor are converted to an audio, visual, or tactile signal by software and/or circuitry in a controller or microprocessor to warn an operator if he/she has inadvertently moved the device while performing a procedure with it.

In embodiments of the method the sensor is attached to a handle of the device.

In embodiments of the method output signals from the sensor are converted to an audio, visual, or tactile signal by software and/or circuitry in a controller or microprocessor to warn an operator if the patient has moved while the operator is performing a procedure with the device.

In embodiments of the method the sensor is an accelerometer.

In embodiments of the method the output signals from one or more of the sensors attached to either a flexible or rigid insertion shaft are processed by software and/or circuitry in a controller or microprocessor to measure the distance that the shaft moves into or out of a lumen relative to a known starting point.

In embodiments of the method the output signals from two sensors attached at two different parts of a flexible or rigid insertion shaft are processed by software and/or circuitry in a controller or microprocessor to measure the relative angle between the two parts.

In embodiments of the method the output signals from several sensors located at different locations along the entire length of a flexible or rigid insertion shaft are processed by software and/or circuitry in a controller or microprocessor to produce information relative to the location and orientation of each of the sensors from which a three dimensional image of the device can be created.

In embodiments of the method the output signals from a magnetic compass on a distal end of the insertion shaft of an endoscope are processed by software and/or circuitry in a controller or microprocessor to navigate the endoscope towards a location within a lumen or cavity inside the body by placing a magnet on the outside surface of the body of the patient.

In a second aspect the invention is a system for accurately determining the location and orientation of a part of an elongated insertion shaft of an endoscope-like device or of an object located on or in the elongated insertion shaft when the elongated insertion shaft is inserted into a lumen or cavity of a human or animal body or into the interior of a manufactured object such that direct visual observation of the part of the elongated insertion shaft or of the object located on or in the elongated insertion shaft is blocked. The system comprises:
a) one or more rotation, position, motion, or angle sensors attached at one or more locations on the device;
b) a controller or microprocessor, which contains software and/or circuitry that are configured to allow the calculation of motion, orientation, or rotation information from the digital or analog signals produced by the sensors;
c) a display unit configured to generate audio or tactile signals and visual images that provide the operator with accurate positional information regarding different parts of the device relative to other parts of the device and to the outside world; and
d) a wired or wireless communication network configured to transfer output signals from the sensors to the controller or microprocessor and the accurate positional information from the controller or microprocessor to the display unit.

In embodiments of the system the sensors are selected from the group comprising: accelerometers, digital gyroscopes, solid state magnetic compasses, angle sensors, and RFID tags.

In embodiments of the system the device is one of an endoscope, a laparoscope, a catheter, and a delivery system, the device configured to be used in a medical application.

In embodiments of the system the device is a borescope or an endoscope, the device being configured to be used in an industrial application.

All the above and other characteristics and advantages of the invention will be further understood through the following illustrative and non-limitative description of embodiments thereof, with reference to the appended drawings.

### BRIEF DESCRIPTION OF THE DRAWINGS

FIG. 1 shows a typical prior art endoscope.

### DETAILED DESCRIPTION OF EMBODIMENTS OF THE INVENTION

A technical problem addressed by the present invention is how to accurately determine the location and orientation of a part of an elongated insertion shaft of a device or of an object located on or in the elongated insertion shaft when the elongated insertion shaft is inserted into a lumen or cavity of a human or animal body or into the interior of a manufactured object such that direct visual observation of the part of the elongated insertion shaft or of the object is blocked. This problem arises in many applications. In industry instruments such as borescopes and endoscopes are used to inspect inter alia the interiors of engines and turbines, and the interior of castings and machined parts. They are also used for inspection in the building industry and many other fields of industry. In medicine instruments such as endoscopes, laparoscopes, catheters, and other types of delivery system are used for inspection, diagnosis, treatment, and delivery of items such as drugs and stents to a particular location within the body.

Another technical problem addressed by the present invention that specifically arises in medical applications is detecting movement of the instrument caused by instability of the hand of the user holding and manipulating the levers, etc. on the handle section.

The present invention provides solutions to these problems that will be illustrated herein below for medical endoscopes; however the solutions described herein can be used with any of the other devices mentioned above and similar devices used in medical and industrial applications.

The solution to the above mentioned problems provided herein is to attach one or more rotation, position, motion, or angle sensors to the endoscope. The sensors can be, for example accelerometers, digital gyroscopes, solid state magnetic compasses, angle sensors and sensors that can be used for triangulation such as RFID tags.

These sensors output either a digital or analog signal proportional to their orientation. The signal is sent using either a wired or wireless communication network to a controller or microprocessor, which, depending on the type of sensor, contains software and/or electronic circuitry that allows the calculation of motion, orientation, or rotation. The output of the controller or microprocessor is sent to a display unit, where an audio signal is generated or a visual image is displayed that provides the operator with accurate positional information regarding different parts of the device relative to other parts of the endoscope and to the outside world.

The output signals of one of these sensors, for example an accelerometer attached to the handle of the endoscope, can be converted to an audio, visual, or tactile signal to warn an operator that he is inadvertently moving the endoscope while concentrating on performing a procedure. Similarly an accelerometer on the shaft of the endoscope, for example on the distal tip, can give a warning if the patient moves suddenly. This embodiment is a very practical and useful addition to any endoscope or laparoscope, whether flexible or rigid.

The output signals from one or more of these sensors, for example an accelerometer attached to the shaft of either a flexible or rigid endoscope or laparoscope can be processed to measure the distance that the shaft moves into or out of a lumen relative to a known starting point. The distance measurement can be either relative to the anatomy of the patient, for example to the teeth, gastroesophageal junction, skin surface, or anus or relative to his surroundings.

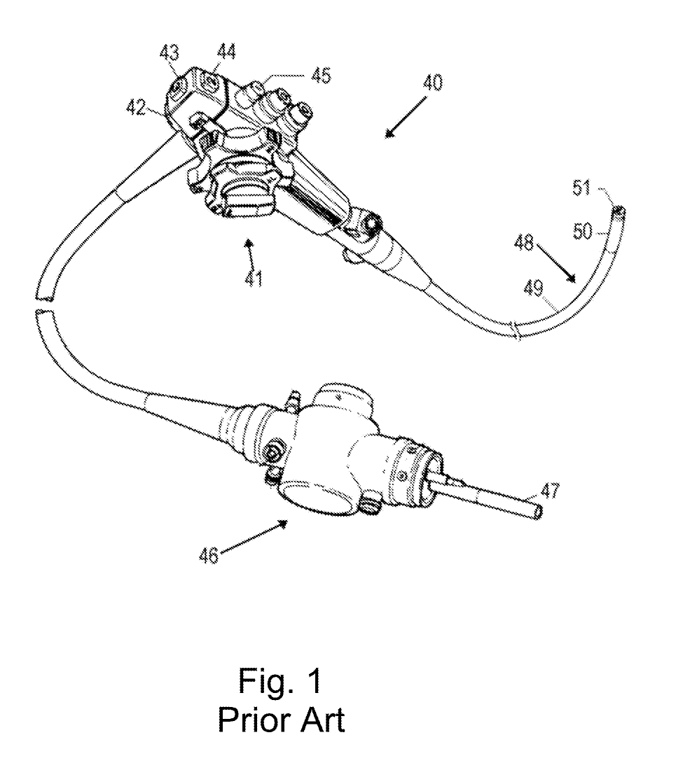

The output signals from two of these sensors attached at different parts of the endoscope can be used to measure the relative angle between the two parts. For example, in an endoscope similar to the one shown in FIG. 1 that was developed by the applicant of this patent application for the treatment of GERD, a staple carrying cartridge component of a stapling device is located inside the esophagus in the flexible portion 49 and the anvil component of the stapling device is located on the face of the distal tip 51 located within the stomach of a patient. The output signals from one of the sensors of the type mentioned above located on or adjacent the cartridge and a second sensor located on or adjacent the anvil could be used to aid in bringing the two components of the stapling device into proper working alignment.

The output from several sensors located at different locations along the entire length of the shaft can be converted by the processor into a three dimensional image of the device.

A magnetic compass, for example on the distal tip, can be used to navigate the endoscope towards a location within a lumen or cavity inside the body by placing a magnet on the outside surface of the body of the patient to mark the location, or can be used to follow a magnet moved on the surface of the body of the patient, or the signals from the magnetic compass can be used to determine the location of the distal tip relative to a stationary magnet outside of the patient.

Although embodiments of the invention have been described by way of illustration, it will be understood that the invention may be carried out with many variations, modifications, and adaptations, without exceeding the scope of the claims.

1. A method to accurately determine the location and orientation of a part of an elongated insertion shaft of an endoscope-like device or of an object located on or in the elongated insertion shaft when the elongated insertion shaft is inserted into a lumen or cavity of a human or animal body or into the interior of a manufactured object such that direct visual observation of the part of the elongated insertion shaft or of the object located on or in the elongated insertion shaft is blocked, the method comprising:
a. attaching one or more rotation, position, motion, or angle sensors at one or more locations on the device;
b. sending via a wired or wireless communication network digital or analog signals produced by the sensors to a controller or microprocessor;
c. employing software and/or circuitry in the controller or microprocessor to determine information relating to motion, orientation, or rotation from the digital or analog signals produced by the sensors;
d. sending the information relating to motion, orientation, or rotation from the controller or microprocessor via a wired or wireless communication network to a display unit configured to generate audio and tactile signals and visual images that provide the operator with accurate positional information regarding different parts of the device relative to other parts of the device and to the outside world.

2. The method of claim 1, wherein the sensors are selected from the group comprising: accelerometers, digital gyroscopes, solid state magnetic compasses, angle sensors and RFID tags.

3. The method of claim 1, wherein the device is one of an endoscope, a laparoscope, a catheter, and a delivery system, the device configured for use in a medical application.

4. The method of claim 1, wherein the device is a borescope or an endoscope, the device configured for use in an industrial application.

5. The method of claim 1, wherein output signals from the sensor are converted to an audio, visual, or tactile signal by software and/or circuitry in a controller or microprocessor to warn an operator if he has inadvertently moved the device while performing a procedure with it.

6. The method of claim 5, wherein the sensor is attached to a handle of the device.

7. The method of claim 3, wherein output signals from the sensor are converted to an audio, visual, or tactile signal by software and/or circuitry in a controller or microprocessor to warn an operator if the patient has moved while the operator is performing a procedure with the device.

8. The method of claim 7, wherein the sensor is an accelerometer.

9. The method of claim 1, wherein the output signals from one or more of the sensors attached to either a flexible or rigid insertion shaft are processed by software and/or circuitry in a controller or microprocessor to measure the distance that the shaft moves into or out of a lumen relative to a known starting point.

10. The method of claim 1, wherein the output signals from two sensors attached at two different parts of a flexible or rigid insertion shaft are processed by software and/or circuitry in a controller or microprocessor to measure the relative angle between the two parts.

11. The method of claim 1, wherein the output signals from several sensors located at different locations along the entire length of a flexible or rigid insertion shaft are processed by software and/or circuitry in a controller or microprocessor to produce information relative to the location and orientation of each of the sensors from which a three dimensional image of the device can be created.

12. The method of claim 3, wherein the output signals from a magnetic compass on a distal end of the insertion shaft of an endoscope are processed by software and/or circuitry in a controller or microprocessor to navigate the endoscope towards a location within a lumen or cavity inside the body by placing a magnet on the outside surface of the body of the patient.

13. A system for accurately determining the location and orientation of a part of an elongated insertion shaft of an endoscope-like device or of an object located on or in the elongated insertion shaft when the elongated insertion shaft is inserted into a lumen or cavity of a human or animal body or into the interior of a manufactured object such that direct visual observation of the part of the elongated insertion shaft or of the object located on or in the elongated insertion shaft is blocked, the system comprising:
a. one or more rotation, position, motion, or angle sensors attached at one or more locations on the device;
b. a controller or microprocessor, which contains software and/or circuitry that are configured to allow the calculation of motion, orientation, or rotation information from the digital or analog signals produced by the sensors;
c. a display unit configured to generate audio or tactile signals and visual images that provide the operator with accurate positional information regarding different parts of the device relative to other parts of the device and to the outside world; and
d. a wired or wireless communication network configured to transfer output signals from the sensors to the controller or microprocessor and the accurate positional information from the controller or microprocessor to the display unit.

14. The system of claim 13, wherein the sensors are selected from the group comprising: accelerometers, digital gyroscopes, solid state magnetic compasses, angle sensors, and RFID tags.

15. The system of claim 13, wherein the device is one of an endoscope, a laparoscope, a catheter, and a delivery system, the device configured to be used in a medical application.

16. The system of claim 13, wherein the device is a borescope or an endoscope, the device being configured to be used in an industrial application.
